(19) United States
(12) Patent Application Publication
(10) Pub. No.: US 2025/0277765 A1
(43) Pub. Date: Sep. 4, 2025

(54) Analysis of Source Rocks in Pyrolysis Experiments

(71) Applicant: Saudi Arabian Oil Company, Dhahran (SA)

(72) Inventors: Poorna Srinivasan, Houston, TX (US); Estefania M. Endara Arguello, Brookshire, TX (US); Shannon L. Eichmann, Katy, TX (US)

(21) Appl. No.: 18/591,635

(22) Filed: Feb. 29, 2024

(51) Int. Cl. G01N 25/14; G01N 1/28; G01N 23/2251; G01N 30/12; G01N 33/24

(52) U.S. Cl. CPC G01N 25/145; G01N 1/286; G01N 23/2251; G01N 30/12; G01N 33/241; G01N 2001/2873; G01N 2030/125

(57) ABSTRACT

The apparatus includes a pyrolysis chamber configured to receive and hold within an interior space a volume of an aqueous fluid and to maintain a pressure within the interior space for a duration of an experimental period when a removable head is in a closed position. The apparatus includes a sample basket assembly comprising a plurality of baskets attached to the removable head by one or more rods. Each basket is configured to receive one or more source rock samples. The sample basket assembly is configured such that, when the removable head is in the closed position and the one or more source samples are disposed within each basket, the samples suspended in a substantially stationary position above the floor.

### TECHNICAL FIELD

This disclosure relates generally to systems and methods to evaluate a source rock sample from a subterranean reservoir.

### BACKGROUND

Petroleum potential of subterranean sources can be evaluated using various laboratory methods. Hydrous pyrolysis utilizes a sealed chamber to subject a rock sample to a controlled pressure and temperature environment under aqueous conditions simulating the natural conditions of the source rock. This technique can be used to simulate the natural generation of petroleum fluid from organic-rich source rocks.

### SUMMARY

Certain aspects of the subject matter herein can be implemented as an apparatus. The apparatus includes a pyrolysis chamber with a floor and walls defining an interior space and a removable head. The pyrolysis chamber is configured to receive and hold within the interior space a volume of an aqueous fluid and to maintain a pressure within the interior space for a duration of an experimental period when the removable head is in a closed position. The apparatus further includes a heater configured to heat the fluid when the fluid is present in the interior space for the duration of the experimental period and a sample basket assembly comprising a plurality of baskets attached to the removable head by one or more rods. Each basket is configured to receive one or more source rock samples. The sample basket assembly is configured such that, when the removable head is in the closed position and the one or more source samples are disposed within each basket, the one or more samples are for the duration of the experimental period suspended in a substantially stationary position above the floor while permitting contact of the one or more source samples with the aqueous fluid, and removal of the removable head from the pyrolysis chamber upon completion of the experimental period removes the plurality of baskets from the interior space such that the one or more samples can be retrieved from the plurality of baskets.

Certain aspects of the subject matter herein can be implemented as a method. The method includes determining a physical or chemical characteristic of a first source rock sample by individually imaging, as a first imaging instance, the first source rock sample and, after the first imaging instance, disposing the first source rock sample in a first basket of a sample basket assembly, the sample basket assembly attached by one or more rods to a removable head of a pyrolysis chamber of a pyrolysis apparatus, the pyrolysis chamber comprising a floor and walls defining an interior space. The method further includes determining a physical or chemical characteristic of second source rock sample by individually imaging, as a second imaging instance, the first source rock sample and, after the second imaging instance, disposing the second source rock sample in a second basket of the sample basket assembly. The method further includes at least partially filling the interior space with a volume of an aqueous fluid and attaching the removable head to the pyrolysis chamber, thereby suspending the first source rock sample and the second source rock sample in the interior space in a position above the floor. With the removable head in the closed position, a pressure and heat are applied to the interior space for a duration of an experimental period. The sample basket assembly affixes the first source rock sample and the second rock sample substantially stationary in the position for the duration of the experimental period while permitting contact of the first source rock sample and the second rock sample with the aqueous fluid. The method further includes, after the experimental period, detaching the removable head from the pyrolysis chamber. After the detaching, the first source rock sample and the second source rock sample are retrieved from the first basket and the second basket, respectively. The method further includes individually imaging, as a third imaging instance, the first source rock sample to determine a change in the physical or chemical characteristic of the first source rock sample, and individually imaging, as a fourth imaging instance, the second source rock sample to determine a change in the physical or chemical characteristic of the second source rock sample.

### DESCRIPTION OF DRAWINGS

FIGS. 1A and 1B are schematic illustrations of an apparatus for conducting pyrolysis experiments in accordance with embodiments of the present disclosure.

FIGS. 2A and 2B are schematic illustrations showing greater detail of a sample basket assembly and its configuration when a removable head is in its open and closed configuration with respect to a pyrolysis chamber in accordance with embodiments of the present disclosure.

FIG. 3 is a schematic illustration showing a sample basket assembly in greater detail in accordance with embodiments of the present disclosure.

FIG. 4 is a process flow diagram of a method 400 of analyzing source rocks for petroleum potential and other related geochemical characteristics in accordance with embodiments of the present disclosure.

### DETAILED DESCRIPTION

High temperature, high pressure batch reaction experiments are used for various geological applications, including, but not limited to, petroleum generation, carbon sequestration, and subsurface hydrogen storage. Hydrous pyrolysis is one type of experiment that can be performed in such a reactor, and this technique is used to simulate the natural generation of petroleum-like products from source rocks. Hydrous pyrolysis generates bitumen and expelled oil from an organic-rich source rock through temperature-controlled experiments in the presence of water. Typical experiments are run between 270-365° C. and last for 72 hours. If running kinetic studies, the time interval can vary between ˜12-300 hours. The goal of using this technique is to artificially mature a source rock from the immature window up to the gas maturity window. At each heating stage, expelled oil and the remaining source rock are analyzed to ascertain maturity and compositional differences. This type of data can be used as input parameters in basin models, helping scientists understand how oil is generated from a source rock in a basin, and what areas should be drilled next during exploration initiatives.

Analytical data can be acquired before and after each experiment, enabling correlations between the original immature source rock and the subsequent artificially heated sample. Maturity and compositional differences obtained before and after the reactor experiments are typically acquired using analytical instruments like a gas chromatograph (GC) and gas chromatograph mass spectrometer (GCMS). However, these instruments can only analyze oils and soluble bitumen that have been extracted using solvents. Obtaining maturity, compositional, and physical data through visual techniques (e.g., microspectrophotometer, petrographic microscope, scanning electron microscope, etc.) can be quite difficult to achieve. This is due to the simple design of the reactor vessel. In most reactor experiments, source rocks are initially crushed to grain sizes between 0.5-2 cm. These rock chips are then mixed with deionized water and merely placed loosely inside of the vessel for the experiments. Because the rock chips can move around the vessel during the experiments and when subsequent retrieval is attempted, it is nearly impossible to image the same rock chip before and after the experiment. Therefore, obtaining data on parameters like pore size distribution, permeability, fracturing, and elemental distributions can be difficult or impossible to achieve. While thermoplastic or glass liners have been designed to hold and contain the rock chips and water at the bottom of the reactor vessel, the extreme pressure and temperature of typical pyrolysis experiments can oxidize and disintegrate the liners. Furthermore, rock samples held either loosely in the chamber or held in liners at the bottom or sides of a chamber can stick to the liners or chamber walls, causing uneven circulation of fluids around the samples during pyrolysis and potentially causing fracturing and other damage to the samples when removal from the chamber is attempted.

In accordance with an embodiment of the present disclosure, an improved apparatus and method for sample management and analysis is disclosed. The apparatus utilizes a sample basket assembly using a plurality of individual baskets comprised of a metallic alloy resistant to high temperatures and pressures. In this way, data be acquired using geochemical techniques for the hydrocarbons expelled from the rock samples and also using imagery techniques for the individual rock samples themselves (e.g., for changes in physical and chemical characteristics of the source rock samples themselves such as maturity, compositional differences of organic matter, porosity, fractures, and elemental distributions). Because the samples are held individually in a proper static position in the baskets and individually retrieved, the user can ensure that the comparisons of experimental data before and after pyrolysis are made using same individual samples. This integration of data can aid in fully understanding how oil is generated in a source rock for basin modeling applications.

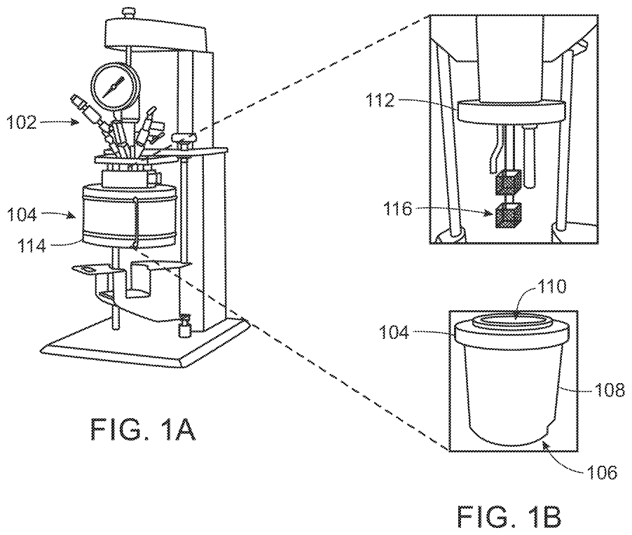

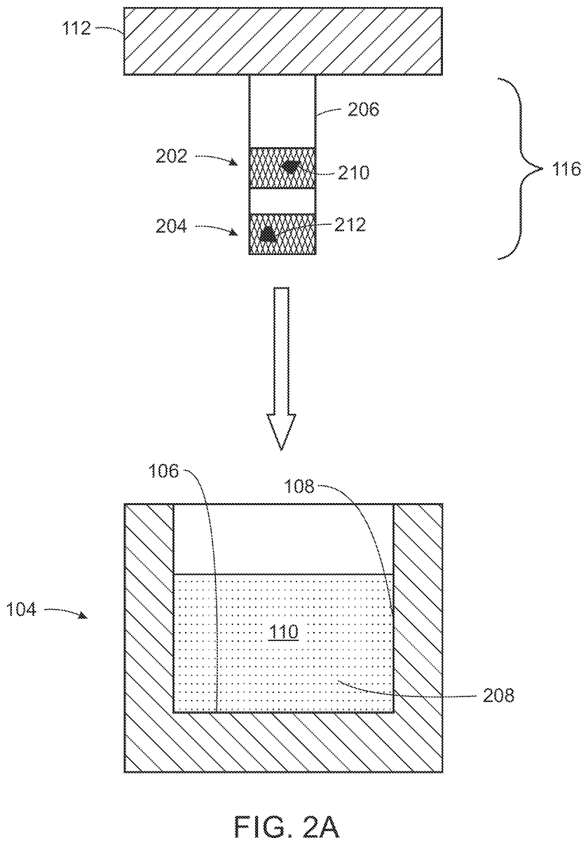

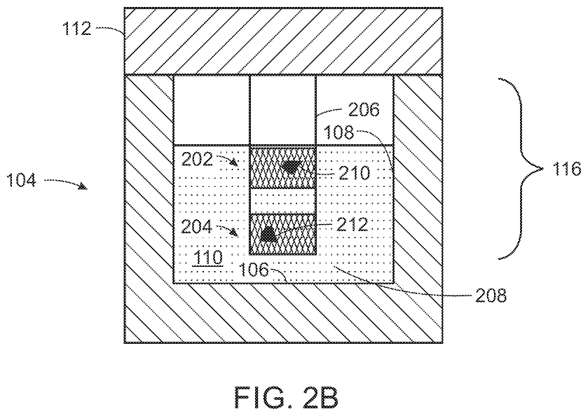

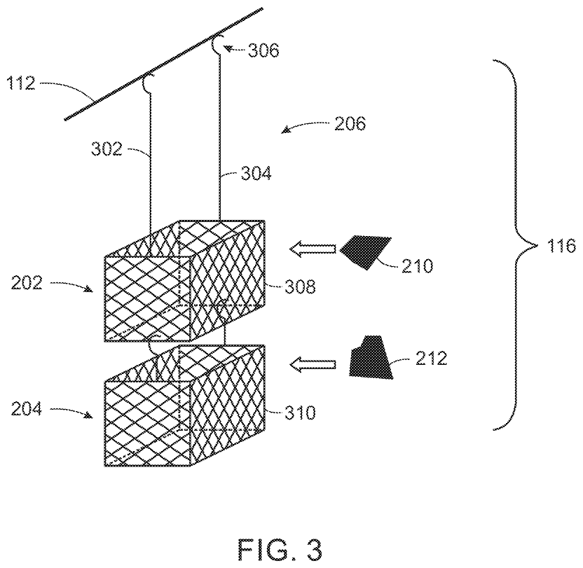

FIGS. 1A and 1B are schematic illustrations of an apparatus for conducting pyrolysis experiments in accordance with embodiments of the present disclosure. Apparatus 102 includes a pyrolysis chamber 104 which in turn includes a floor 106 and walls 108. In some embodiments, the floor and walls are separate components joined together; in other embodiments, the floor and walls are a single component. Floor 106 and walls 108 in turn define an interior space 110. Interior space 110 can be enclosed by attaching removable head 112 (for example, with a threaded connection) to form a pressure seal. A sample basket assembly 116 is attached to removable head 112, and is described in greater detail in FIGS. 2A-2B and FIG. 3.

Apparatus 102 further includes a heater 114 configured to heat fluid in the interior space 110 when the fluid is present in the interior space for the duration of the experimental period. Apparatus 102 can further include one or more pressure inlets and outlets, and various sensors, gauges, and meters for sensing and displaying the temperature, pressure, and other parameters within interior space 110 and in connection with other aspects of the apparatus. When the removable head is attached and a seal is formed, the apparatus is configured to hold within the interior space 110 a volume of an aqueous fluid and to maintain a desired pressure and temperature within the interior space for a duration of a pyrolysis experiment.

FIGS. 2A and 2B are schematic illustrations showing greater detail of sample basket assembly 116 and its configuration when removable head 112 is in its open and closed configurations in accordance with embodiments of the present disclosure. In the illustrated embodiment, sample basket assembly 116 includes a first metallic basket 202 and a second metallic basket 204. In some embodiments a sample basket assembly can include a greater or lesser number of baskets. Baskets 202 and 204 are attached to removable head 112 by one or more rods 206, each metallic basket configured to such that each one or more source rock samples (for example, samples 210 and 212) can be readily disposed within the baskets when removable head 112 is not attached to chamber 104 (FIG. 2A). When the removable head is in the closed position (FIG. 2B) the basket assembly ensures that the samples, for the duration of the experimental period, are suspended in a substantially stationary position above the floor within interior space 110. An aqueous fluid 208 (for example, an aqueous liquid) can be placed within interior space 110. During the pyrolysis experiment, the temperature in the interior space 110 can be high enough (for example, approximately 270 degrees or greater) to partially or completely vaporize aqueous fluid 208. Upon completion of the experiment, removal of the removable head 112 from the pyrolysis chamber removes the plurality of metallic baskets from the interior space such that the one or more samples can be readily retrieved from the plurality of metallic baskets.

FIG. 3 is a schematic illustration showing sample basket assembly 116 in greater detail in accordance with embodiments of the present disclosure. In the illustrated embodiment, wherein the rods 206 comprise a pair of rods 302 and 304. Rods 302 and 304 are attached at their respective ends to removable head 112 by, for example, a hook 306 or other suitable attachment. The other ends of rods 302 and 304 are attached to the sample baskets 202 and 204. More specifically, in the illustrated embodiment, rod 302 is attached to one side of baskets 202 and 204 is attached to the other side of sample baskets 202 and 204.

In some embodiments, rods 302 and 304 are flexible wires; in other embodiments, rods 302 and 304 are rigid rods. Baskets 202 and 204 and rods 302 and 304 in some embodiments are made of a nickel-chromium-molybdenum alloy such as Hastelloy C276 (UNS N10276), which can withstand temperatures up to 1600° C. and is resistant to corrosion that can occur within a pressure vessel when conducting pyrolysis experiments, particularly when such experiments include high-sulfur source rocks that can generate H₂S, which acidifies water and causes corrosion to metals such as carbon steels.

The wire gridding of the baskets can be large enough to allow the aqueous fluid (liquid or vapor) to flow through and be in contact with the samples during the experiment, while being small enough that no sample or sample pieces can fall out of the basket. In some embodiments, the baskets are sized (and otherwise configured) such that a rock sample disposed therein that has been cut (sized) for a sample holder of an imaging apparatus (such as an SEM) is held substantially immobilized relative to the removable head. By holding the sample substantially immobile relative to the removable head during the experiment and also as the head is attached to and detached from the chamber, fracturing or other damage to the sample can be reduced or eliminated, and no further cutting or dimensioning of the sample is necessary before the sample is placed into the imaging apparatus sample holder. For example, insertion of a sample into an SEM imaging apparatus may require that the sample be cut to approximately 1 cm (length) by 1 cm (width) by 0.25 cm (height). In order to hold such a sample immobile in the basket, the basket can have dimensions of approximately 1.5 cm×1.5 cm×1.5 cm with gridding approximately 3 mm wide. The baskets can include doors 308 and 310 which can be open so that samples (for example, samples 210 and 212) can be easily placed inside the individual respective baskets and the doors closed to aid in immobilizing the samples within the basket. Although FIG. 3 shows two baskets, each attached to a pair of rods and each holding one sample, in other embodiments a sample assembly can include a different number of baskets, one or more of which may hold one (or more than one) sample, and a greater or lesser number of rods. For example, a basket assembly in some embodiments can include three baskets, each configured to hold a respective group of two or more rock samples. In some embodiments, in addition to the samples held in the baskets, some samples can be placed loosely in the chamber.

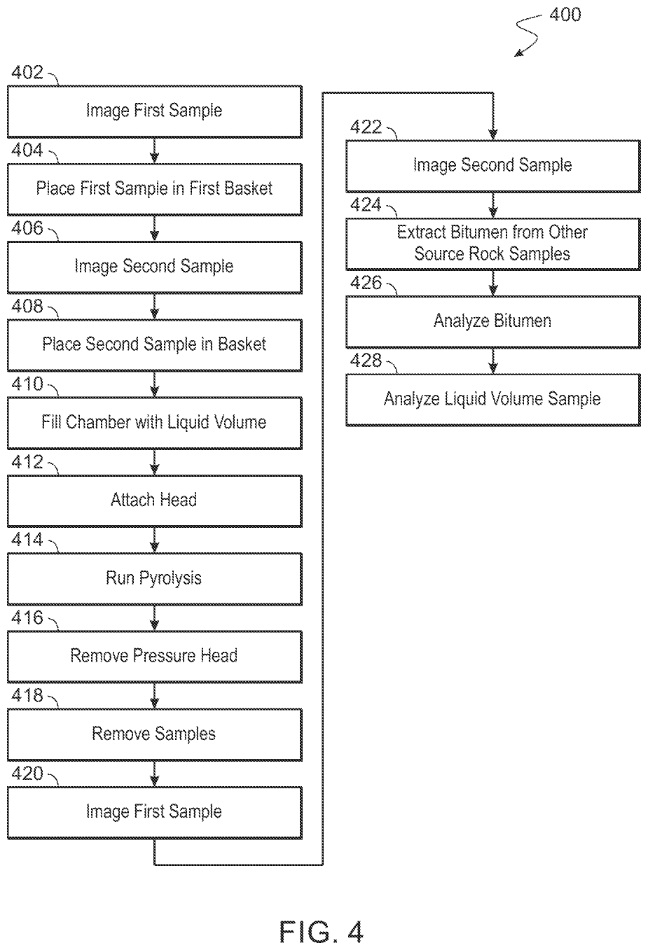

FIG. 4 is a process flow diagram of a method 400 of analyzing source rocks for petroleum potential and other related geochemical characteristics in accordance with embodiments of the present disclosure. Method 400 will be described in reference to system 100 described in reference to the above figures. The method begins at step 402 in which one or more physical or chemical characteristics of an individual source rock sample are determined using scanning electron microscopy (SEM), petrographic microscopy, micro-spectrophotometry, or other suitable imaging technique. The physical or chemical characteristics can include, for example, maturity, composition, or other characteristics. In preparation for the imaging, rock can be cut with a diamond saw and ion-milled such that they are a suitable size and shape for the imaging apparatus and for placement in a sample basket. For example, in some embodiments, if a sample basket is approximately 1.45 cm in length and width, a sample can be cut to approximately 1 cm (length) by 1 cm (width) by 0.25 cm (height). In some embodiments, the sample can be etched to distinguish it from other rock samples used in the experiment and to aid in for consistent orientation in the imaging apparatus for imaging before and after a pyrolysis experiment.

Proceeding to step 404, the sample can be placed in one of the sample baskets of the sample basket assembly. At step 406, sample preparation and imaging can similarly be conducted on a second sample that, at step 408, can be placed in another of the sample baskets of the sample basket assembly. In some embodiments, one sample is disposed in each basket. In some embodiments, a greater number of samples (for example, two or three or more) are disposed in one or both baskets. In some embodiments, a sample basket assembly can include a greater number of baskets (for example, three baskets). In some embodiments, a sample basket assembly has only one basket (such that steps 406 and 408 can be omitted).

At step 410, a volume of an aqueous liquid such as deionized water is added to the interior space of the pyrolysis chamber, at least partially filling it. In some embodiments, the volume or aqueous liquid is approximately 30 to 40 milliliters. Proceeding to step 412, the removable head is attached to the pyrolysis chamber, such that the source rock samples are suspended and held in a stationary position above the floor within the chamber. In some embodiments, at this step, in addition to the rock samples in the baskets, additional rock samples are also placed loosely in the interior space (not in the baskets). In some embodiments, the total weight of all of rock samples combined can be approximately 30 grams.

Proceeding to step 414, with the removable head in the closed position and scaled, pressure and heat are applied to the interior space for a duration of the pyrolysis portion of the experiment. The sample basket assembly suspended by the rods affixes the first source rock sample and the second rock sample substantially stationary in the position for the duration, away from the sides and floor of the chamber of the experimental period. An experimental run can have, for example a duration of 12 to 300 hours at a temperature of 270-365° C.). Some runs can be for a greater or lesser time at a greater or lesser temperature. In some runs, the pressure and/or temperature are varied during the run.

Proceeding to step 416, after the experimental period, after the allotted time of the pyrolysis run has been completed for its full duration, the chamber is allowed to cool to room temperature and the removable head is removed from the chamber, allowing easy access to the baskets and allowing for the rock samples to be individually retrieved. At step 418, after removal from the baskets, the samples can be washed with toluene (or another aromatic solvent) to remove any oil that is adsorbed to the surface of the rock pieces. This adsorbed oil can be analyzed by geochemical instruments such as a gas chromatograph and gas chromatograph mass spectrometer. The samples can be placed onto sample holders (or into epoxy plugs) for imaging using the same imaging techniques and apparatus as before the pyrolysis run (for example, SEM, petrographic microscope, and/or microspectrophotometer) to determine any changes caused by the pyrolysis run in the physical or chemical characteristics of the samples. In some embodiments, the rock samples can be etched prior to placement in the baskets, such that the samples can be placed in the imaging apparatus in the same orientation as the pre-pyrolysis imaging, further enabling accurate and precise comparisons.

In some embodiments, the method proceeds to step 424 in which the remaining rock pieces can undergo solvent extraction using dichloromethane (or an equivalent solvent) to remove soluble bitumen. The solvent-extracted bitumen can at step 426 be analyzed by geochemical instruments such as a GC and GCMS. Similarly, at step 427, any hydrocarbons expelled from the samples into the surrounding liquid can be removed with a pipette and likewise geochemically analyzed using GC, GCMS, or other suitable techniques.

In this disclosure, “approximately” or “substantially” mean a deviation or allowance of up to 10 percent (%) and any variation from a mentioned value is within the tolerance limits of any machinery used to manufacture the part. Likewise, “about” can also allow for a degree of variability in a value or range, for example, within 10%, within 5%, or within 1% of a stated value or of a stated limit of a range.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “0.1% to about 5%” or “0.1% to 5%” should be interpreted to include about 0.1% to about 5%, as well as the individual values (for example, 1%, 2%, 3%, and 4%) and the sub-ranges (for example, 0.1% to 0.5%, 1.1% to 2.2%, 3.3% to 4.4%) within the indicated range. The statement “X to Y” has the same meaning as “about X to about Y,” unless indicated otherwise. Likewise, the statement “X, Y, or Z” has the same meaning as “about X, about Y, or about Z,” unless indicated otherwise.

A number of implementations have been described. Nevertheless, it will be understood that various modifications may be made without departing from the spirit and scope of the disclosure. For example, example operations, methods, or processes described herein may include more steps or fewer steps than those described. Further, the steps in such example operations, methods, or processes may be performed in different successions than that described or illustrated in the figures. Accordingly, other implementations are within the scope of the following claims.

### EXAMPLES

In a first aspect, an apparatus includes a pyrolysis chamber with a floor and walls defining an interior space and a removable head. The pyrolysis chamber is configured to receive and hold within the interior space a volume of an aqueous fluid and to maintain a pressure within the interior space for a duration of an experimental period when the removable head is in a closed position. The apparatus further includes a heater configured to heat the fluid when the fluid is present in the interior space for the duration of the experimental period and a sample basket assembly comprising a plurality of baskets attached to the removable head by one or more rods. Each basket is configured to receive one or more source rock samples. The sample basket assembly is configured such that, when the removable head is in the closed position and the one or more source samples are disposed within each basket, the one or more samples are for the duration of the experimental period suspended in a substantially stationary position above the floor while permitting contact of the one or more source samples with the aqueous fluid, and removal of the removable head from the pyrolysis chamber upon completion of the experimental period removes the plurality of baskets from the interior space such that the one or more samples can be retrieved from the plurality of baskets.

In a second aspect in accordance with the first aspect, each basket of the plurality of baskets is a metallic basket.

In a third aspect in accordance with the first or second aspects, each basket of the plurality of baskets comprise nickel-chromium-molybdenum alloy.

In a fourth aspect in accordance with any of the first to third aspects, the one or more rods comprise nickel-chromium-molybdenum alloy.

In a fifth aspect in accordance with any of the first to fourth aspects, the one or more rods comprise metal wires.

In a sixth aspect in accordance with any of the first to fifth aspects, the one or more rods comprise rigid metal rods.

In a seventh aspect in accordance with any of the first to sixth aspects, the one or more rods comprise a pair of rods, and wherein a first end of a first rod of the pair of rods and a first end of a second rod of the pair of rods are attached to the removable head and a second end of the first rod of the pair of rods is attached to a first side of a first basket of the plurality of baskets and a second end of the second rod of the pair of rods is attached to a second side of the first basket of the plurality of baskets.

In an eighth aspect in accordance with any of the first to seventh aspects, at least one basket of the plurality of baskets is dimensioned such that a sample sized for a sample holder of an imaging apparatus is substantially immobilized relative to the removable head if the sample sized for the sample holder is disposed within the at least one basket.

In a ninth aspect in accordance with any of the first to eighth aspects, the sample holder of the imaging apparatus is configured to hold a sample of approximately 1 cm by 1 cm by 0.25 cm and the at least one basket is approximately 1.5 cm×1.5 cm×1.5 cm.

In a tenth aspect, a method includes determining a physical or chemical characteristic of a first source rock sample by individually imaging, as a first imaging instance, the first source rock sample and, after the first imaging instance, disposing the first source rock sample in a first basket of a sample basket assembly, the sample basket assembly attached by one or more rods to a removable head of a pyrolysis chamber of a pyrolysis apparatus, the pyrolysis chamber comprising a floor and walls defining an interior space. The method further includes determining a physical or chemical characteristic of second source rock sample by individually imaging, as a second imaging instance, the first source rock sample and, after the second imaging instance, disposing the second source rock sample in a second basket of the sample basket assembly. The method further includes at least partially filling the interior space with a volume of an aqueous fluid and attaching the removable head to the pyrolysis chamber, thereby suspending the first source rock sample and the second source rock sample in the interior space in a position above the floor. With the removable head in the closed position, a pressure and heat are applied to the interior space for a duration of an experimental period. The sample basket assembly affixes the first source rock sample and the second rock sample substantially stationary in the position for the duration of the experimental period while permitting contact of the first source rock sample and the second rock sample with the aqueous fluid. The method further includes, after the experimental period, detaching the removable head from the pyrolysis chamber. After the detaching, the first source rock sample and the second source rock sample are retrieved from the first basket and the second basket, respectively. The method further includes individually imaging, as a third imaging instance, the first source rock sample to determine a change in the physical or chemical characteristic of the first source rock sample, and individually imaging, as a fourth imaging instance, the second source rock sample to determine a change in the physical or chemical characteristic of the second source rock sample.

In an eleventh aspect in accordance with the tenth aspect, at least the first imaging instance and the third imaging instance of the imaging instances comprises imaging with an SEM apparatus.

In a twelfth aspect in accordance with the tenth or eleventh aspects, at least one of the imaging instances comprises imaging with a petrographic microscope.

In a thirteenth aspect in accordance with any of the tenth to eleventh aspects, at least one of the imaging instances comprises imaging with microspectrophotometer.

In a fourteenth aspect in accordance with any of the tenth to thirteenth aspects, the method further includes, before the first imaging instance, etching a pattern in the first source rock, and wherein the first imaging instance and the third imaging instance comprise orienting the first rock sample in a sample holder of an imaging apparatus based in part on the etched pattern.

In a fifteenth aspect in accordance with any of the tenth to fourteenth aspects, the method further includes, the experimental period, determining a geochemical characteristic of a hydrocarbon liquid expelled during the experimental period from one or more source rock samples disposed in the interior space.

In a sixteenth aspect in accordance with any of the tenth to fifteenth aspects, the determining the geochemical characteristic comprises analyzing the hydrocarbon liquid with a gas chromatograph.

In a seventeenth aspect in accordance with any of the tenth to sixteenth aspects, the first source rock sample and the second rock sample comprise two of three or more source rock samples disposed in the interior space for the duration of the experimental period, and further comprising, after the experimental period, extracting bitumen from one or more of the three or more source rock samples other than the first source rock sample and the second source rock sample.

In an eighteenth aspect in accordance with any of the tenth to seventeenth aspects, the plurality of baskets comprise nickel-chromium-molybdenum alloy.

In a nineteenth aspect in accordance with any of the tenth to eighteenth aspects, at least the first basket is dimensioned such that a sample sized for a sample holder of an imaging apparatus is substantially immobilized relative to the removable head if the sample sized for the sample holder is disposed within the first basket, and further comprising, before the first imaging instance, cutting the first source rock sample such that the first source rock sample is sized for the sample holder.

In a twentieth aspect in accordance with any of the tenth to nineteenth aspects, the sample holder of the imaging apparatus is configured to hold a sample of approximately 1 cm by 1 cm by 0.25 cm and the first basket is approximately 1.5 cm×1.5 cm×1.5 cm.

1. An apparatus comprising:
a pyrolysis chamber comprising a floor and walls defining an interior space and a removable head, the pyrolysis chamber configured to receive and hold within the interior space a volume of an aqueous fluid and to maintain a pressure within the interior space for a duration of an experimental period when the removable head is in a closed position;
a heater configured to heat the fluid when the fluid is present in the interior space for the duration of the experimental period; and
a sample basket assembly comprising a plurality of baskets attached to the removable head by one or more rods, each basket configured to receive one or more source rock samples, the sample basket assembly configured such that:
when the removable head is in the closed position and the one or more source samples are disposed within each basket, the one or more samples are for the duration of the experimental period suspended in a substantially stationary position above the floor while permitting contact of the one or more source samples with the aqueous fluid; and
removal of the removable head from the pyrolysis chamber upon completion of the experimental period removes the plurality of baskets from the interior space such that the one or more samples can be retrieved from the plurality of baskets.

2. The apparatus of claim 1, wherein each basket of the plurality of baskets is a metallic basket.

3. The apparatus of claim 1, wherein each basket of the plurality of baskets comprise nickel-chromium-molybdenum alloy.

4. The apparatus of claim 1, wherein the one or more rods comprise nickel-chromium-molybdenum alloy.

5. The apparatus of claim 1, wherein the one or more rods comprise metal wires.

6. The apparatus of claim 1, wherein the one or more rods comprise rigid metal rods.

7. The apparatus of claim 1, wherein the one or more rods comprise a pair of rods, and wherein a first end of a first rod of the pair of rods and a first end of a second rod of the pair of rods are attached to the removable head and a second end of the first rod of the pair of rods is attached to a first side of a first basket of the plurality of baskets and a second end of the second rod of the pair of rods is attached to a second side of the first basket of the plurality of baskets.

8. The apparatus of claim 1, wherein at least one basket of the plurality of baskets is dimensioned such that a sample sized for a sample holder of an imaging apparatus is substantially immobilized relative to the removable head if the sample sized for the sample holder is disposed within the at least one basket.

9. The apparatus of claim 8, wherein the sample holder of the imaging apparatus is configured to hold a sample of approximately 1 cm by 1 cm by 0.25 cm and the at least one basket is approximately 1.5 cm×1.5 cm×1.5 cm.

10. A method comprising:
determining a physical or chemical characteristic of a first source rock sample by individually imaging, as a first imaging instance, the first source rock sample;
after the first imaging instance, disposing the first source rock sample in a first basket of a sample basket assembly, the sample basket assembly attached by one or more rods to a removable head of a pyrolysis chamber of a pyrolysis apparatus, the pyrolysis chamber comprising a floor and walls defining an interior space;
determining a physical or chemical characteristic of second source rock sample by individually imaging, as a second imaging instance, the first source rock sample;
after the second imaging instance, disposing the second source rock sample in a second basket of the sample basket assembly;
at least partially filling the interior space with a volume of an aqueous fluid;
attaching the removable head to the pyrolysis chamber, thereby suspending the first source rock sample and the second source rock sample in the interior space in a position above the floor;
with the removable head in the closed position, applying a pressure and heat to the interior space for a duration of an experimental period, the sample basket assembly affixing the first source rock sample and the second rock sample substantially stationary in the position for the duration of the experimental period while permitting contact of the first source rock sample and the second rock sample with the aqueous fluid;
after the experimental period, detaching the removable head from the pyrolysis chamber; and
after the detaching:
retrieving the first source rock sample and the second source rock sample from the first basket and the second basket, respectively;
individually imaging, as a third imaging instance, the first source rock sample to determine a change in the physical or chemical characteristic of the first source rock sample; and
individually imaging, as a fourth imaging instance, the second source rock sample to determine a change in the physical or chemical characteristic of the second source rock sample.

11. The method of claim 10, wherein at least the first imaging instance and the third imaging instance of the imaging instances comprises imaging with an SEM apparatus.

12. The method of claim 10, wherein at least one of the imaging instances comprises imaging with a petrographic microscope.

13. The method of claim 10, wherein at least one of the imaging instances comprises imaging with microspectrophotometer.

14. The method of claim 10, further comprising, before the first imaging instance, etching a pattern in the first source rock, and wherein the first imaging instance and the third imaging instance comprise orienting the first rock sample in a sample holder of an imaging apparatus based in part on the etched pattern.

15. The method of claim 10, further comprising, after the experimental period, determining a geochemical characteristic of a hydrocarbon liquid expelled during the experimental period from one or more source rock samples disposed in the interior space.

16. The method of claim 15, wherein the determining the geochemical characteristic comprises analyzing the hydrocarbon liquid with a gas chromatograph.

17. The method of claim 10, wherein the first source rock sample and the second rock sample comprise two of three or more source rock samples disposed in the interior space for the duration of the experimental period, and further comprising, after the experimental period, extracting bitumen from one or more of the three or more source rock samples other than the first source rock sample and the second source rock sample.

18. The method of claim 10, wherein the plurality of baskets comprise nickel-chromium-molybdenum alloy.

19. The method of claim 10, wherein at least the first basket is dimensioned such that a sample sized for a sample holder of an imaging apparatus is substantially immobilized relative to the removable head if the sample sized for the sample holder is disposed within the first basket, and further comprising, before the first imaging instance, cutting the first source rock sample such that the first source rock sample is sized for the sample holder.

20. The method of claim 10, wherein the sample holder of the imaging apparatus is configured to hold a sample of approximately 1 cm by 1 cm by 0.25 cm and the first basket is approximately 1.5 cm×1.5 cm×1.5 cm.
